(19) United States
(12) Patent Application Publication
(10) Pub. No.: US 2016/0375383 A1
(43) Pub. Date: Dec. 29, 2016

(54) Water Filter with Electrical Conductivity Meter

(71) Applicants: Erez Mazor, Givat Avni (IL); Ronen Aharon Cohen, Moza Elit (IL)

(72) Inventors: Erez Mazor, Givat Avni (IL); Ronen Aharon Cohen, Moza Elit (IL)

(21) Appl. No.: 15/258,060

(22) Filed: Sep. 7, 2016

Related U.S. Application Data
(63) Continuation-in-part of application No. 14/082,218, filed on Nov. 18, 2013, now abandoned.

(51) Int. Cl. B01D 36/02; G01N 27/06; B01D 29/11; C02F 1/00; C02F 5/10; B01D 24/10

(52) U.S. Cl. CPC B01D 36/02; C02F 5/10; B01D 24/10; B01D 29/117; C02F 1/001; G01N 27/06; C02F 2303/02; C02F 2209/05; C02F 2209/02; G01N 33/18

(57) ABSTRACT

A water filter that includes a stand-alone housing, a replaceable filter cartridge core, and an electrical conductivity meter that includes a main body and electrodes. The stand-alone housing includes a base into which the replaceable filter cartridge core is designed to be inserted and a cover designed to close and seal the stand-alone housing. The cover includes an internal chamber designed to contain the electrical conductivity meter, a water inlet pipe and a water outlet pipe. The internal chamber has an outward-facing opening that extends until the outer part of the cover and an inward-facing opening that extends to the water outlet opening. The conductivity meter is designed to be inserted into the internal chamber in a way that its main body may be visible through the outward-facing opening and the electrodes come in contact with water flowing in the water outlet pipe.

### FIELD OF THE INVENTION

The present invention refers to a water filter that includes an electrical conductivity meter designed to test the water quality in real time.

### BACKGROUND OF THE INVENTION

Water filtering is a process of cleaning water from materials, chemicals and contaminations. The most common objective of water filtering is to render it suitable for drinking. Water filters are usually installed in houses in order to render the running water suitable for drinking. However, people who consume the filtered water are often not sure whether the filters that are installed on the main pipe of their houses indeed clean the water properly. The present invention provides a good and efficient solution for this problem.

US Patent Application No. 2006/0011523A1 (hereinafter “US Application 523”) describes a water filter cartridge that includes a sensor unit with a conductance sensor that is equipped with two electrodes. The housing of the water filter cartridge has an opening or a breach in it through which the sensor unit can be inserted. The sensor unit in US Application 523 is designed to be installed on the filter cartridge through said opening or breach. The electrodes of the unit sensor are then inserted into the water filter cartridge.

The invention described in US Application 523 has several disadvantageous. First, when the need arises to replace the water filter cartridge, the user must uninstall the sensor unit and reinstalled it on the new cartridge. The invention, subject of the present application, describes a permanent housing for a replaceable filter cartridge core that includes a special chamber into which a sensor is installed permanently. When the need arises to replace the replaceable filter cartridge core, the user needs not touch the sensor. Second, US Application 523 refers to a replaceable water filter cartridge, whereas the invention subject of the present application refers to a permanent housing for a replaceable filter cartridge core that includes a special chamber into which a sensor is installed permanently.

### LIST OF DRAWINGS

The intention of the drawings attached to the application is not to limit the scope of the invention and its application. The drawings are intended only to illustrate the invention and they constitute only one of its many possible implementations.

FIG. 1 describes a front sectional view of the water filter (1) that includes a housing, an electrical conductivity meter (3) and a filter cartridge core (4).

FIG. 2 describes a perspective sectional view of the housing (2) that includes a base (21), a cover (22), an internal chamber (23), a water inlet (11), and a water outlet (12).

FIG. 3 describes a perspective sectional view of the filter cartridge core (4) that includes a gauze filter (41), siliphos balls (43), a ceramic filter (44), and a carbonate filter (42).

FIG. 4 describes a perspective view of the electrical conductivity meter (3).

FIG. 5 describes the upper part of the water filter (1) that includes the internal chamber (23) and the first internal chamber (231).

FIG. 6 describes the set (111) that comprises a water filter (1) and two external chambers (232) designed to be installed in the water inlet and outlet (11) (12) or along the pipe near said water inlet and outlet (11) (12).

### THE INVENTION

The term “water filter” in this patent application refers to a filter that comprises a housing containing a filter cartridge core. The filter cartridge core may comprise several filters. As is clear from the drawings attached to the application and from the explanation in this application, the water filter (1) comprises a stand-alone housing (2) designed to house a replaceable filter cartridge core (4). The stand-alone housing (2) includes a base (21) into which the replaceable filter cartridge core (4) is inserted, a cover (22) designed to close and seal the stand-alone housing (2), and an internal chamber (23) designed to contain an electrical conductivity meter (3).

The main objective of the present invention is to provide a water filter that includes an electrical conductivity meter (hereinafter “ECM”) and a replaceable filter cartridge core (hereinafter “FCC”) that includes one or more of the following types of filters: Net filter, carbonate filter, antibacterial ceramic balls filter, and siliphos balls. Another objective of the invention is to provide a FCC that consists of several filters. Another objective of the invention is to provide a FCC that is also aimed to be inserted into the housing that contains the ECM.

The water filter (1) comprises a stand-alone housing (2), an electrical conductivity meter (ECM) (3) and a replaceable filter cartridge core (FCC) (4) that may include one or more of the following filters: gauze filter (41), carbonate filter (42), antibacterial ceramic balls filter (44), and siliphos balls (43).

The stand-alone housing (2) can be shaped like any other housing for standard water filters. The stand-alone housing (2) includes a base (21) into which the FCC (4) is designed to be inserted, a cover (22) designed to close and seal the stand-alone housing (2), and an internal chamber (23) designed to contain the ECM (3). The cover (22) includes a water inlet pipe (11), a water outlet pipe (12) and an internal chamber (23).

The replaceable filter cartridge core (FCC) (4) may contain, as mentioned above, a gauze filter (41), a carbonate filter (42), antibacterial ceramic balls filter (44), and siliphos balls (43). The structure and combination of such filters are known to any average professional expert in this field and therefore there is no need to describe them here in details.

The gauze filter (41) is usually made of materials such as polypropylene and it is designed to remove floating solids, sand, rust and rough dirt from the water. The carbonate filter (42) comprises a chamber that contains active carbonate that removes organic materials and materials that cause bad smell and taste from the water. Siliphos balls (43) are commonly ball shaped and they are designed to prevent scale formation and thus protect plumbing and appliances that operated with water. The siliphos is diluted in the water and prevents the minerals, which cause the scale, from accumulating in the plumbing and pipes, despite the fact that the water is sometimes hot and/or standing.

The ceramic filter (44) comprises a chamber that contains ceramic balls made of materials such as SiO₂, Al₂O₃ and TiO₂. The ceramic filter (44) is designed to remove micro-organisms, toxic minerals and bacteria out of the water.

The ECM (3) is a known component and is used in many fields. In general, the ECM (3) comprises several anodes that are inserted into a water pipe, and a processor unit that measures the conductivity of the water between those anodes. The conductivity, along with the weighted water temperature indicates the amount of chemicals and dirt in water. The ECM (3) is inserted in the stand-alone housing (2) near the water outlet, i.e., in the water after they flow through the FCC (4). The more dirt and contamination the water contains, the lower its conductivity, and vice versa. The ECM (3) is equipped with a display panel (31) that shows the water condition, which depends on the level of chemicals and contamination in the water. When this level exceeds a pre-determined value, a warning appears on the display panel (31), indicating that the water is not clean and that the FCC (4) must to be replaced, treated or cleaned. The display panel (31) can be an independent and separated unit, i.e., it does not have to be integrally connected to the ECM (3). In such a case, the ECM (3) and the display panel (31) both include a contact and when these two contacts are connected the results are displayed on the display panel (31).

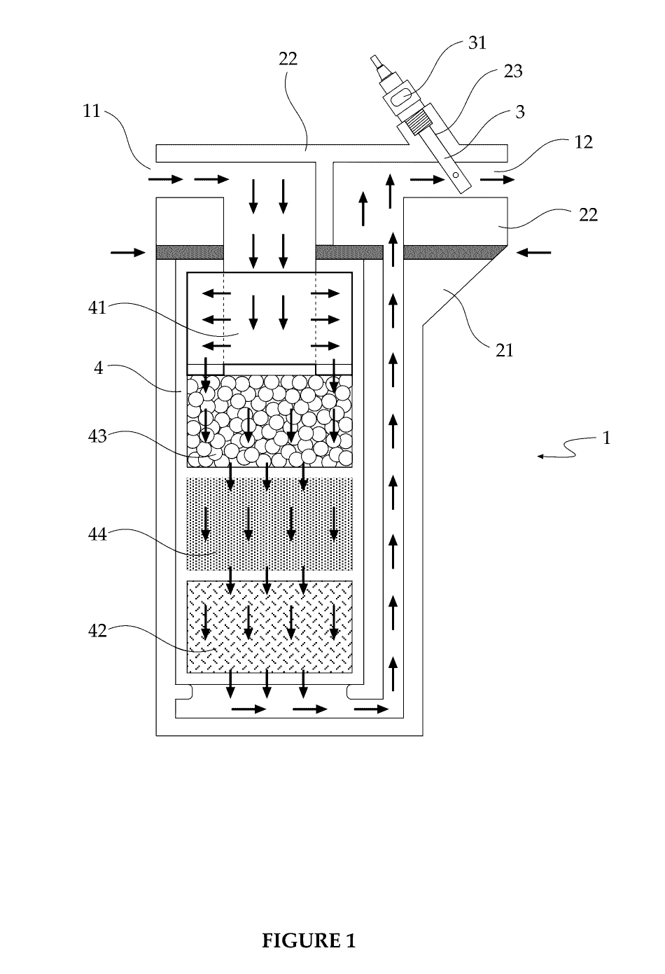

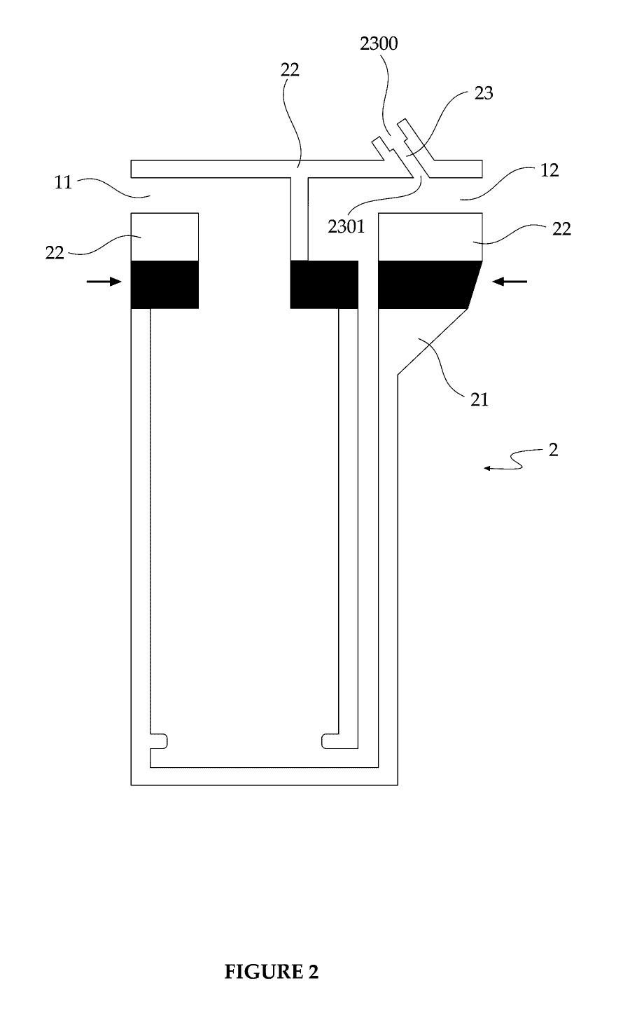

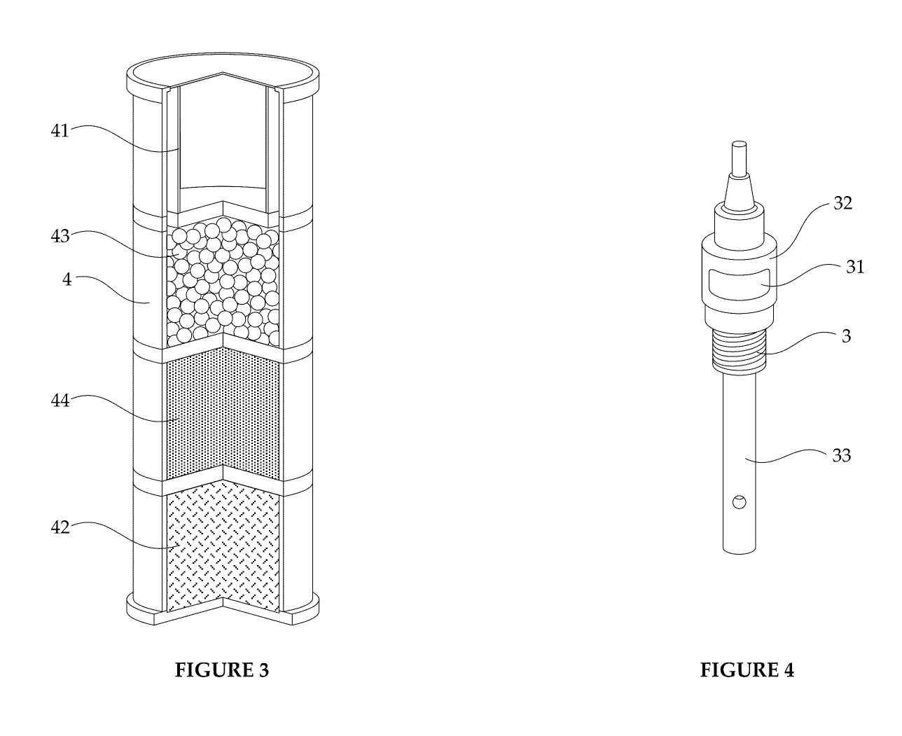

The water flow path through the water filter (1) is shown in FIG. 1. The water flows through the water inlet pipe (11), the gauze filter (41), the siliphos balls (43) chamber, the ceramic filter (44), the carbonate filter (43), to the water outlet pipe (12) into which the ECM (3) is inserted.

The innovation of the water filter (1) subject of the present invention lies in the fact that it contains the FCC (4) which is capable of cleaning and removing chemicals and organic materials from water and monitoring and determining whether the ECM (4) is fulfilling its purpose, whether it is functioning, and when it requires replacement, treatment or cleaning. Thus, the user may purchases a single product unit that consists of a filter housing, a filter core, and electrical conductivity meter with a display panel, which enables water cleaning and purification as well as real time monitoring and controlling of the process.

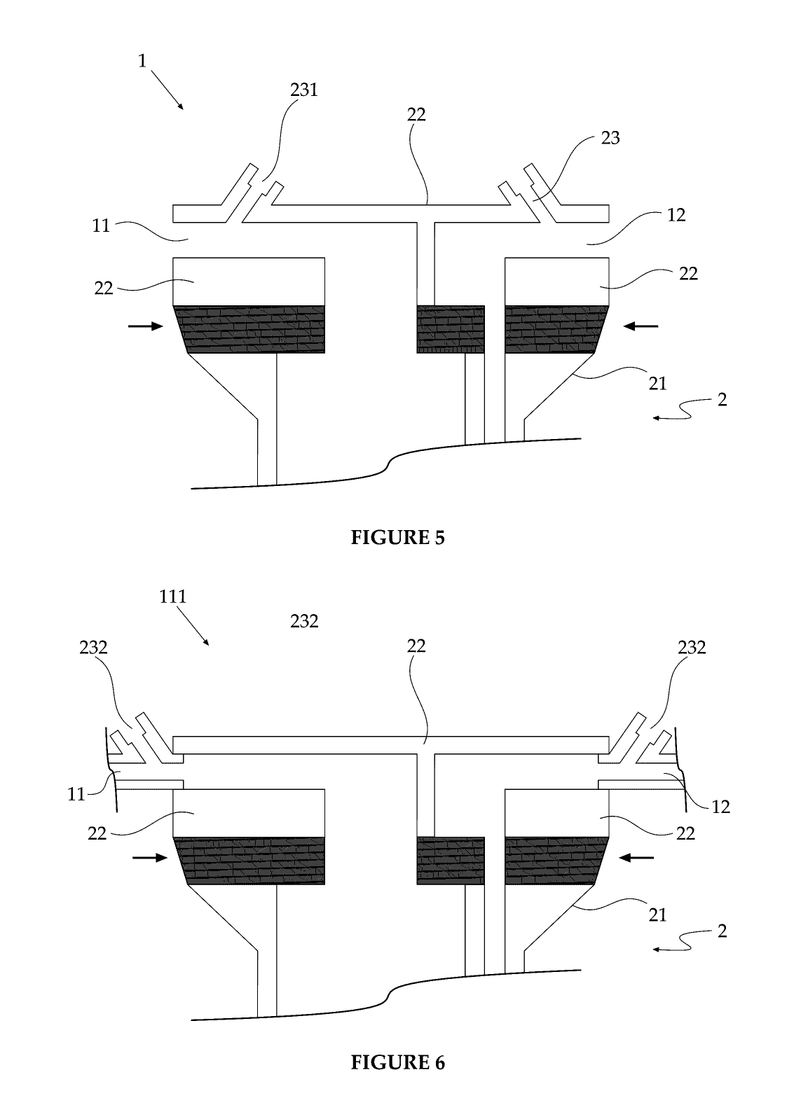

The second embodiment of the water filter (1) includes the stand-alone housing (2), as described above, as well as a first internal chamber (231) with an ECM (3) in the water inlet pipe (11). The ECM (3) located in the internal chamber (23), which is located near the water outlet pipe (12) monitors the quality of the water after it is filtered while the ECM (3) in the first internal chamber (231), which is located near the water inlet pipe (11), monitors the quality of the water before it is filtered. Thus, the user can receive information about the filter efficiency. FIG. 5 describes the upper part of the water filter (1) of the second embodiment.

The third embodiment of the present invention refers to a set (111) that comprises a water filter (1) and one or two separable external chambers (232) designed to be installed in the water inlet and outlet pipes (11) (12) or along the pipe near said water inlet and outlet pipes (11) (12). The ECM (3) is designed to be inserted into said external chambers (232). When the set (111) includes two separable external chambers (232), the second external chamber is designed to be installed along the pipe (100) near the water inlet pipe (11) or unto the water inlet pipe (11) itself. The second external chamber (232) is installed along the pipe near the water inlet pipe (11) or in said inlet pipe (11) itself in order to determine filter efficiency. FIG. 6 describes the set (111) that comprises the water filter (1) with two separable external chambers (232), which are installed near the water inlet and outlet pipes (11) (12).

The term “chemicals and dirt” as used above and as used in this application refers to a wide variety of dirt, salts, blood, metals that permeate into groundwater from industrial sewage and so on. The water filter (1), subject matter of this invention is intended for domestic and industrial use as well as for water systems in laboratories and machines.

The water filter (1) comprises a stand-alone housing (2), a replaceable filter cartridge core (4) for water treatment, and an electrical conductivity meter (3). The stand-alone housing (2) comprises a base (21) into which the replaceable filter cartridge core (4) is designed to be inserted and a cover (22) designed to close and seal the stand-alone housing (2). The cover (22) includes the internal chamber (23) designed to contain the electrical conductivity meter (3), the water inlet pipe (11) and the water outlet pipe (12). The conductivity meter (3) includes a main body (32) and electrodes (33). The internal chamber (23) has an outward-facing opening (2300) and an inward-facing opening (2301). The outward-facing opening (2300) extends until the outer part of the cover (22) and the inward-facing opening (2301) extend to the water outlet pipe (12) which is an area inside the water filter where water flows outside the water filter. The conductivity meter (3) is designed to be inserted into the internal chamber (23) whereby the main body (32) may be visible through the outward-facing opening (2300) while the electrodes (33) come in contact with water flowing inside the water filter.

What we claim is:

1. A water filter, comprises: a stand-alone housing, a replaceable filter cartridge core for water treatment, and an electrical conductivity meter; wherein said stand-alone housing comprises a base into which said replaceable filter cartridge core is designed to be inserted and a cover designed to close and seal said stand-alone housing; wherein said cover includes an internal chamber designed to contain said electrical conductivity meter, a water inlet pipe and a water outlet pipe; wherein said conductivity meter includes a main body and electrodes; wherein the internal chamber has an outward-facing opening and an inward-facing opening; wherein said outward-facing opening extends until the outer part of said cover; wherein said inward-facing opening extends to the water outlet opening; whereby said conductivity meter is designed to be inserted into said internal chamber and whereby said main body may be visible through said outward-facing opening while said electrodes come in contact with water flowing in the water outlet pipe.

2. A stand-alone housing of a water filter that is designed to contain a replaceable filter cartridge core for water treatment, comprises: a base into which said replaceable filter cartridge core is designed to be inserted and a cover designed to close and seal said stand-alone housing; wherein said cover includes an internal chamber designed to contain an electrical conductivity meter, a water inlet pipe and a water outlet pipe; wherein said conductivity meter includes a main body and electrodes; wherein the internal chamber has an outward-facing opening and an inward-facing opening; wherein said outward-facing opening extends until the outer part of said cover; wherein said inward-facing opening extend to the water outlet pipe; whereby said conductivity meter is designed to be inserted into said internal chamber and whereby said main body may be visible through said outward-facing opening while said electrodes come in contact with water flowing in the water outlet pipe.
